SEQUENCE LISTING (12 sequences)

SEQ ID NO: 1 - DNA, 21 bp, Homo sapiens
  source 1..21: mol_type = genomic DNA
actcctacatattagcattaa

SEQ ID NO: 2 - DNA, 21 bp, Homo sapiens
  source 1..21: mol_type = genomic DNA
actcctacatgttagcattaa

SEQ ID NO: 3 - DNA, 65 bp, Homo sapiens
  source 1..65: mol_type = genomic DNA
ctgttaatgctaatcgtgataggggtttttgcctccaactgactcctacatattagcattaacag

SEQ ID NO: 4 - DNA, 21 bp, Mus musculus
  source 1..21: mol_type = genomic DNA
actcctacctgttagcattaa

SEQ ID NO: 5 - DNA, 65 bp, Homo sapiens
  source 1..65: mol_type = genomic DNA
ctgttaatgctaatcgtgataggggtttttgcctccaactgactcctacatgttagcattaacag

SEQ ID NO: 6 - DNA, 65 bp, Homo sapiens
  source 1..65: mol_type = genomic DNA
ctgttaatgctaatcatgataggggtttttgcctccaactgactcctacatattagcattaacag

SEQ ID NO: 7 - DNA, 65 bp, Homo sapiens
  source 1..65: mol_type = genomic DNA
ctgttaatgctaatcgtgataggggtttttgcccccaactgactcctacatattagcattaacag

SEQ ID NO: 8 - DNA, 65 bp, Homo sapiens
  source 1..65: mol_type = genomic DNA
ctgttaatgctaatcgtgataggggtttttgcctccaactgactcctacgtattagcattaacag

SEQ ID NO: 9 - DNA, 65 bp, Homo sapiens
  source 1..65: mol_type = genomic DNA
ctgttaatgctaatcgtgataggggtttttgcctccaactaactcctacatattagcattaacag

SEQ ID NO: 10 - DNA, 65 bp, Homo sapiens
  source 1..65: mol_type = genomic DNA
ctgttaatgctaatcgtgataggggtttttgcctccaactgactcctatatattagcattaacag

SEQ ID NO: 11 - DNA, 70 bp, Homo sapiens
  source 1..70: mol_type = genomic DNA
ctgttaatgctaatcgtgataggggtttttgcctccaactgactcctacatattagcattaacagtgtac

SEQ ID NO: 12 - DNA, 250 bp, synthetic construct
  source 1..250: mol_type = other DNA
gtaggtcgacgtttaaacgcgatcgcagatctgcatgtcgataacgcagagactcaacaccctgtttattgatgctgatgaatgacagctcgtaattcagtgactgactggccaggttcatctgctgtaataacgccccggacgcgggagtggccgaggcgttagcagagaataacaggctacctgtcactaatgacatggcaaaccaaagttgcttcaaagcttgatgaattgaagcttttttgaattc

(19) United States
(12) Patent Application Publication
(10) Pub. No.: US 2026/0337606 A1
(43) Pub. Date: Oct. 29, 2026

(54) Therapeutic Use of the miR155 SNP rs377265631

(71) Applicant: LEIBNIZ-INSTITUT FÜR IMMUNTHERAPIE (LIT), Regensburg (DE)

(72) Inventors: Luca GATTINONI, Regensburg (DE); Dragana SLAVKOVIC-LUKIC, Regensburg (DE); Neal LACEY, Los Angeles, CA (US); Yun JI, Germantown, MD (US)

(21) Appl. No.: 19/105,944

(22) PCT Filed: Aug. 30, 2023
(86) PCT No.: PCT/EP2023/073808

§ 371 Date: Feb. 24, 2025

(30) Foreign Application Priority Data
Sep. 2, 2022 (EP) 22193672.7

(51) Int. Cl. C12N 15/113; A61P 35/00

(52) U.S. Cl. CPC C12N 15/113; A61P 35/00; C12N 2310/141

(57) ABSTRACT

The present invention relates to the therapeutic use of a single nucleotide polymorphism (SNP) of miR155. The SNP has a multifunctional beneficial effect on immune cells, such as T cells and can be recombinantly employed in the treatment of diseases, such as cancer.

### STATEMENT REGARDING FUNDING

Parts of this invention were made with governmental support through the National Institutes of Health. The Government has certain rights in the invention.

### BACKGROUND

MicroRNAs (miRNAs; miRs) are small non-coding RNAs that control gene expression of a broad set of target genes based on sequence complementarity. By binding to the 3′ untranslated regions (3′UTR) of target mRNAs, miRNAs regulate gene expression and potentially enable control of multiple gene targets within the same or distinct signaling pathways (Nat Rev Immunol. 2016; 16(5):279-294; Nat Rev Drug Discov. 2010; 9(10):775-789). Many miRNAs are dysregulated in cancer, cardiovascular and autoimmune diseases (FEBS J. 2018; 285(20):3695-3716). Genomic mutations, deletion, or changes in the key enzymes in miRNA biogenesis may all lead to alterations in miRNA levels. (Nat Rev Cancer. 2011; 11(12):849-864; Nat Rev Cancer. 2009; 9(4):293-302).

microRNAs are involved in the normal functioning of eukaryotic cells. Dysregulation of micro RNAs may lead to disease and for many microRNAs a role in pathogenesis or disease has been reported (FEBS J. 2018; 285(20):3695-3716). Diseases associated with the dysregulation of miRNAs include cancer (Nature. 2012; 482(7385): 347-355), heart diseases (Heart. 2015; 101:921-928), kidney diseases (Nature Reviews Nephrology 2015; 11:23-33), diseases of the nervous system (Brain. 2011; 134(Pt12): 3578-3589), obesity (Cells. 2019; 8(8):859) hemostatic diseases (JTH. 2015; 13(2): 170-81) and others. Depending on the type of dysregulation, various therapeutic approaches are possible. For certain diseases, in particular diseases which are caused or associated with the up-regulation of a microRNA, it might be beneficial to inhibit the functioning or the expression of the microRNA (Blood. 2018; 132(Supplement 1): 2903). For other diseases, in particular diseases which are caused or associated with the down-regulation of a microRNA, it might be beneficial to recombinantly express the microRNA or to add therapeutic agents that lead to an increased expression of the microRNA (Proc Natl Acad Sci USA. 2015; 112(2):476-81, Blood. 2015; 125(22):3377-3387).

One specific microRNA is miR155. miR155 has been used in numerous approaches, including the use of miR155 as a diagnostic or prognostic marker, therapeutic use of miR155 by inhibiting or blocking its activity, and therapeutic use by expressing miR155 in tissues or cells (Carcinogenesis. 2020; 41(1):2-7, Blood. 2018; 132(Supplement 1): 2903, Blood. 2011; 118(21): 2728, Int J Mol Sci. 2020; 21(16):5834). The biology of miR155 is multi-facetted. miR155 is a key regulator of Treg homeostasis (Immunity. 2009; 30(1): 80-91). miR155 also promotes pro-inflammatory macrophages while inhibiting the polarization of anti-inflammatory macrophages (Cell Mol Immunol. 2009; 6(5):343-352). miR155 also enhances DC cytokine production and co-stimulatory function (Science. 2007; 316(5824): 608-611). miR155 also promotes B cell proliferation, survival, germinal center formation, plasma cell differentiation and antibody production (Immunity. 2007; 27(6): 847-859). miR155 also promotes Th1, Th17, Tfh differentiation while inhibiting type 2 polarization (Immunity. 2010; 33(4): 607-619). miR155 also enhances NK cells proliferation, chemotaxis and effector functions (Blood 2013; 121(16): 3126-3134; Plos One 2020; 15(2): e0225820.). Lastly, miR-155 promotes CD8+ T cell effector responses against viruses and tumors (2013 Apr. 18; 38(4):742-53; Nat Immunol. 2013 June; 14(6):593-602).

WO2007/127190 describes the development of a transgenic mouse model in which the mice express miR155. WO2007/127190 does however not contemplate any therapeutic use of miR155. WO2009/026576 discloses certain nucleic acids, so called external guide sequences (EGS), that may be used to target and thereby down-regulated the activity of miR-155. WO2010/135714 relates to the modulation of genes involved in adipocyte expression via compositions including miR155. WO2011/029903 relates to the therapeutic use of miR's via enrichment of microRNAs in blood preparations of patients. One of the miRs is miR155. WO2014/059248 relates to the enhancement of anti-cancer immunity through expression of miR155 in specific T cells. A similar concept is disclosed in WO2014/066137. WO2016/077574 discloses inhibitors of miR-155 to increase atrial natriuric (ANP) levels for the treatment of cardiovascular diseases. WO2017/182580 relates to the treatment of production-related disorders with, among others, miR155. WO2018/177746 relates to the treatment of polycystic ovary syndrome with, among others, miR155. WO2019/227260 relates to mammalian virus-mediated miR expression, including miR155. WO2020/002430 relates to the stimulation of mesenchymal stem cells with miRs, such as miR155. WO2020/221821 relates to the treatment of cognitive disorders with, among others, miR155. CN112481218 describes a pig mIR-155 vector system. CN113337544 relates to a retroviral vector expressing a CAR and a microRNA, wherein said microRNA is, among others, miR155. None of the aforementioned references however mentions or otherwise anticipates nor suggests SNP4.

For many genes single-nucleotide polymorphisms (SNPs) are described. SNPs refer to variants of a gene in which a single nucleotide in the genome is substituted. SNPs have also been described for microRNAs. The SNP rs377265631 (SNP4) is one of many SNPs that were described for miR155. SNP4 carries an A-to-G variation compared to wildtype miR155:

wildtype miR155
(SEQ ID No. 1)
ACTCCTACATATTAGCATTAA
rs377265631 (SNP4)
(SEQ ID No. 2)
ACTCCTACATGTTAGCATTAA

The function or role of rs377265631 was unknown prior to the present invention. Herein it is demonstrated for the first time that overexpression of rs377265631 has a beneficial effect effect in the treatment of disease, such as cancer. This effect surprisingly surmounts the effect obtained with the corresponding with type microRNA.

### SUMMARY OF THE INVENTION

The present disclosure relates to a specific SNP of miR155 which surprisingly has been found to be useful in the treatment of various diseases, such as cancer. In certain embodiments, the present disclosure relates to a recombinant nucleic acid comprising the nucleic acid sequence of SEQ ID No. 2 or SEQ ID No. 5 for use in medicine. In certain embodiments said use in medicine is the use in the treatment of cancer. In certain embodiments, said use in the treatment of cancer is the treatment of a melanoma, NSCLC, sarcoma or a HPV-associated cancer.

In certain embodiments, said nucleic acid comprising the nucleic acid sequence of SEQ ID No. 2 or SEQ ID No. 5 is operably linked to a promoter. In certain embodiments, said recombinant nucleic acid is encoded on an expression vector. In certain embodiments, said expression vector is a viral vector or a plasmid. In certain embodiments, recombinant nucleic acid is an isolated or purified nucleic acid.

In certain embodiments, the present disclosure relates to a recombinant immune cell comprising a nucleic acid of SEQ ID No. 2 or SEQ ID No. 5. In certain embodiments, recombinant immune cell is a T cell. In certain embodiments, said T cell is a CD8-positive T cell. In certain embodiments, said CD8-positive T cell is a tumor infiltrating lymphocyte (TIL) or a peripheral blood lymphocyte (PBL) isolated from a patient afflicted with cancer. In certain embodiments, said immune cell further comprises a nucleic acid encoding a chimeric antigen receptor or a T cell receptor.

In certain embodiments, the nucleic acid comprising the nucleic acid sequence of SEQ ID No. 2 or SEQ ID No. 5 is located within the coding region of said chimeric antigen receptor or said T cell receptor, such as the intracellular part of the chimeric antigen receptor or the T cell receptor. In certain embodiments, said chimeric antigen receptor or said T cell receptor is specific for a cancer antigen. In certain embodiments, said immune cell is an isolated or purified immune cell. In certain embodiments, said immune cell is a human immune cell.

In certain embodiments, the present disclosure relates to a population of cells comprising at least one of aforementioned immune cells. In certain embodiments, the present disclosure relates to a composition comprising at least one of aforementioned immune cells and a carrier therefor, preferably a pharmaceutically acceptable carrier.

In certain embodiments, the present disclosure relates to a recombinant immune cell, a population of cells or a composition as described herein for use in medicine. In certain embodiments, said use in medicine is the use in the treatment of cancer. In certain embodiments, said cancer is a melanoma, NSCLC, sarcoma or a HPV-associated cancer.

### Definitions

The terms “miR”, “miRNA” and “microRNA” as used herein refers to the unprocessed or processed RNA transcript from a miR gene. miRs are capable of regulating the expression of genes through interacting with messenger RNA molecules (mRNA), DNA or proteins. Typically, microRNAs are composed of nucleic acid sequences of about 19-25 nucleotides (bases) and are found in mammalian cells. Mature microRNA molecules are single stranded RNA molecules processed from double stranded precursor transcripts that form local hairpin structures. The hairpin structures are typically cleaved by RNAses, such as Dicer, Argonaut, or RNAse 111, into an active 19-25 nucleotide RNA molecule. The unprocessed miR gene transcript is also called an “miR precursor”, and typically comprises an RNA transcript of about 70-100 nucleotides in length. This active 19-25 nucleotide RNA molecule is also called the “processed” miR gene transcript or “mature” miRNA. It is to be understood that the term “miR” as used herein can include one or more of miR-oligonucleotides, including mature miRs, pre-miRs, pri-miRs, or a miR seed sequence. In certain embodiments, the miRs may be modified to enhance delivery. Various sources for miRNA (miR) information is available (e.g. the Sanger Institute, miRbase, TargetSCAN, miRDB). The term microRNA as used herein incorporates both the duplex (i.e., double stranded miRs) and single stranded miRs (i.e., mature miRs) in both the 5′ to 3′ direction and complementary strand in the 3′ to 5′ direction.

The terms “miR155”, “miRNA155” and “microRNA155” as used herein refer to a specific microRNA with the GeneID 406947. The unprocessed precursor, pre-miR155, has the following sequence:

(SEQ ID NO. 3)
CTGTTAATGCTAATCGTGATAGGGGTTTTTGCCTCCAACTGACTCCTACA
TATTAGCATTAACAG

Processed, mature miR155 has the following sequence:

(SEQ ID NO. 1)
ACTCCTACATATTAGCATTAA

Orthologues of miR155 exist in many other species:

TABLE 1

| | | |
|---|---|---|
| Human | ACTCCTACATATTAGCATTAA | SEQ ID No. 1 |
| Bonobo | ACTCCTACATATTAGCATTAA | SEQ ID No. 1 |
| Chimpanzee | ACTCCTACATATTAGCATTAA | SEQ ID No. 1 |
| Gorilla | ACTCCTACATATTAGCATTAA | SEQ ID No. 1 |
| Orang utan | ACTCCTACATGTTAGCATTAA | SEQ ID No. 2 |
| Gibbon | ACTCCTACATGTTAGCATTAA | SEQ ID No. 2 |
| Vervet-AGM | ACTCCTACATGTTAGCATTAA | SEQ ID No. 2 |
| Crab-eating | ACTCCTACATGTTAGCATTAA | SEQ ID No. 2 |
| macaque | | |
| Macaque | ACTCCTACATGTTAGCATTAA | SEQ ID No. 2 |
| Olive baboon | ACTCCTACATGTTAGCATTAA | SEQ ID No. 2 |
| Gelada | ACTCCTACATGTTAGCATTAA | SEQ ID No. 2 |
| Marmoset | ACTCCTACATGTTAGCATTAA | SEQ ID No. 2 |
| Mouse lemur | ACTCCTACATGTTAGCATTAA | SEQ ID No. 2 |
| Prairie vole | ACTCCTACATGTTAGCATTAA | SEQ ID No. 2 |
| Mouse | ACTCCTACCTGTTAGCATTAA | SEQ ID No. 4 |
| Algerian mouse | ACTCCTACCTGTTAGCATTAA | SEQ ID No. 4 |
| Rabbit | ACTCCTACATGTTAGCATTAA | SEQ ID No. 2 |
| Cow | ACTCCTACATGTTAGCATTAA | SEQ ID No. 2 |
| Goat | ACTCCTACATGTTAGCATTAA | SEQ ID No. 2 |
| Sheep | ACTCCTACATGTTAGCATTAA | SEQ ID No. 2 |
| Pig | ACTCCTACATGTTAGCATTAA | SEQ ID No. 2 |
| Horse | ACTCCTACATGTTAGCATTAA | SEQ ID No. 2 |
| Dog | ACTCCTACATGTTAGCATTAA | SEQ ID No. 2 |
| Dingo | ACTCCTACATGTTAGCATTAA | SEQ ID No. 2 |
| Cat | ACTCCTACATGTTAGCATTAA | SEQ ID No. 2 |
| Leopard | ACTCCTACATGTTAGCATTAA | SEQ ID No. 2 |

The terms “rs377265631” and “SNP4” as used herein refer to a specific SNP of miR155. The unprocessed precursor of SNP4 has the following sequence:

(SEQ ID NO. 5)
CTGTTAATGCTAATCGTGATAGGGGTTTTTGCCTCCAACTGACTCCTACA
TGTTAGCATTAACAG

Processed SNP4 has the following sequence:

(SEQ ID NO. 2)
ACTCCTACATGTTAGCATTAA

Of note, the sequence of SNP4 is identical to the miR155 orthologues of orangutan, gibbon and several other species; see Table 1.

The terms “SNP” and “single nucleotide polymorphism” refer to a polymorphism at a particular position in the genome of a species that varies among a population of individuals, i.e. for which two or more alternative alleles are present in a given population.

The term “cell” as used herein refers to a single cell or a plurality of cells.

The term “host cell” as used herein refers to a cell comprising a nucleic acid and/or a vector. In the context of the present disclosure, the term host cell refers to a cell comprising a nucleic acid and/or a vector encoding a miR, preferably SNP4. Such host cell will express the miR and is suitable to be used in medicine. Preferred host cells of the present invention are eukaryotic host cells, such as immune cells. Particular preferred host cells are T cells, such as CD8-positive T cells.

The term “T cell” as used herein refers to a type of lymphocyte that plays a central role in cell-mediated immunity. T cells, also referred to as T lymphocytes, can be distinguished from other lymphocytes, such as B cells and natural killer cells, by the presence of a T-cell receptor (TCR) on the cell surface. There are several subsets of T cells with distinct functions, including but not limited to, T helper cells, cytotoxic T cells, memory T cells, regulatory T cells and natural killer T cells. In certain embodiments, the T cell is an engineered T cell. In other embodiments, the T cell is a CD8-positive T cell. In yet other embodiments, the T cell is a CAR-T cell.

The terms “CD8” or “cluster of differentiation 8” as used herein refer to a transmembrane glycoprotein (UniProt: P01732) which is present on certain T cells and which serves as a co-receptor for the T-cell receptor (TCR). Along with the TCR, the CD8 co-receptor plays a role in T cell signaling and aiding with cytotoxic T cell-antigen interactions. T cells expressing CD8 are referred to as “CD8-positive T cells”.

The term “CAR” or “chimeric antigen receptor” as used herein refers to an artificial cell surface receptor that is designed to bind to certain proteins on cells, for example cancer cells. When certain CARs are expressed by a T cell, binding of the CAR extracellular binding moiety with a target antigen can activate the T cell. CARs are also known as chimeric T cell receptors or chimeric immunoreceptors. Typical CAR comprise (i) an extracellular domain that includes a moiety that binds a target antigen; (ii) a transmembrane domain; and (iii) an intracellular signaling domain that sends activating signals when the CAR is stimulated by binding of the extracellular binding moiety with a target antigen.

The term “CAR-T cell” as used herein refers to a T cell that has been engineered to express a chimeric antigen receptor.

The terms “Tcell receptor” or “TCR” as used herein refer to a complex of integral membrane proteins that participates in the activation of T cells in response to the binding of an antigen. The TCR is a disulfide-linked membrane-anchored heterodimer normally consisting of the highly variable alpha and beta chains expressed as the part of a complex with the invariant CD3 (cluster of differentiation 3) chain molecules.

The terms “polynucleotide” and/or “nucleic acid sequence” and/or “nucleic acid” as used herein refer to a sequence of nucleoside or nucleotide monomers consisting of bases, sugars and intersugar (backbone) linkages. The term includes DNA and RNA and can be either double stranded or single stranded, and represents the sense or antisense strand. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acids of the present disclosure may be isolated from biological organisms, formed by laboratory methods of genetic recombination or obtained by chemical synthesis or other known protocols for creating nucleic acids.

The terms “modified RNA” or “modified DNA” as used herein refers to a nucleic acid molecule in which one or more of the components of the nucleic acid, namely sugars, bases, and phosphate moieties, are different from that which occurs in nature. Such molecules have at least one modified internucleoside linkage and/or at least one sugar modification and/or at least one base modification compared to a naturally occurring ribonucleotide- or deoxyribonucleotide-based oligonucleotide. A modified internucleoside linkage indicates the presence of a modified version of the phosphodiester which does not occur naturally in RNA and DNA. Examples of internucleoside linkage modifications are and include in particular, phosphoramidate, phosphorodiamidate, phosphorothioate, phosphorodithioate, H-phosphonate, methyl phosphonate and methyl phosphonothioate. A sugar modification indicates the presence of a modified version of the ribosyl moiety as naturally occurring in RNA and DNA (i.e. the furanosyl moiety), such as bicyclic sugars, tetrahydropyrans, morpholinos, 2′-modified sugars, 3′-modified sugars, 4′-modified sugars, 5′-modified sugars, and 4′-substituted sugars. Examples of suitable sugar modifications are known to the skilled person in the art and include, but are not limited to, 2′-O-modified RNA nucleotide residues, such as 2′-O-alkyl or 2′-O-(substituted)alkyl, e.g. 2′-O-methyl, 2′-O-(2-cyanoethyl), 2′-O-(2-methoxy)ethyl (2′-MOE), 2′-O-(2-thiomethyl)ethyl; 2′-O-(haloalkoxy)methyl, e.g. 2′-O-(2-chloroethoxy)methyl (MCEM), 2′-O-(2,2-dichloroethoxy)methyl (DCEM); 2′-O-alkoxycarbonyl, e.g. 2′-0-[2-(methoxycarbonyl)ethyl](MOCE), 2′-O-[2-(N-methylcarbamoyl)ethyl](MCE), 2′-O-[2-(N,N-dimethylcarbamoyl)ethyl](DMCE), in particular a 2′-O-methyl modification or a 2′-O-(2-methoxy)ethyl (2′-MOE). Another important modification includes bridged or bicylic nucleic acid (BNA) modified sugar moieties, such as found in e.g. locked nucleic acid (LNA), xylo-LNA, alpha-L-LNA, beta-D-LNA, cEt (2′-0,4′-C constrained ethyl) LNA, cMOEt (2′-0,4′-C constrained methoxy ethyl) LNA, ethylene-bridged nucleic acid (ENA), hexitol nucleic acid (ETNA), fluorinated HNA (F-HNA), pyranosyl-RNA (p-RNA), 3′-deoxypyranosyl-DNA (p-DNA); or other modified sugar moieties, such as morpholino (PMO), cationic morpholino (PMOPlus) or PMO-X. The term “base modification”, as used herein refers to the modification of a naturally occurring base in RNA and/or DNA (i.e. pyrimidine or purine base). Base modifications include, but are not limited to, a modified version of the natural purine and pyrimidine bases (e.g. adenine, uracil, guanine, cytosine, and thymine), such as hypoxanthine, pseudouracil, pseudothymine, 2-thiopyrimidine (e.g. 2-thiouracil, 2-thiothymine), 2,6-diaminopurine, 5-substituted pyrimidine (e.g. 5-halouracil, 5-methyluracil, 5-methylcytosine) 7-deazaguanine, 7-deazaadenine, 7-aza-2,6-diaminopurine, 8-aza-7-deazaguanine, 8-aza-7-deazaadenine, or 8-aza-7-deaza-2,6-diaminopurine. It is also encompassed that said oligonucleotide comprises more than one, the same or different, internucleoside linkage modification, sugar modification and/or base modification.

The terms “adenine base editor” or “ABE” refer to a base editor that mediates conversion of adenosine-to-guanosine (i.e. A-T to G-C) via an inosine intermediate. Numerous ABE's are known in the art including, but not limited to ABE7.10, ABE 6.3, ABE7.8 and ABE7.9.

The terms “isolated polynucleotide” or “isolated nucleic acid sequence” as used herein refer to a nucleic acid substantially free of cellular material or culture medium when produced by recombinant DNA techniques, or chemical precursors, or other chemicals when chemically synthesized.

The terms “recombinant nucleic acid” or “engineered nucleic acid” as used herein refer to a nucleic acid or polynucleotide that is not found in a biological organism. For example, recombinant nucleic acids may be formed by laboratory methods of genetic recombination (such as molecular cloning) to create sequences that would not otherwise be found in nature. Recombinant nucleic acids may also be created by chemical synthesis or other known protocols for creating nucleic acids. Unless otherwise indicated, the definitions and embodiments described in this and other sections are intended to be applicable to all embodiments and aspects of the present application herein described for which they are suitable as would be understood by a person skilled in the art.

The term “polypeptide” or “protein” as used herein describes a chain of amino acids. A polypeptide or protein of this disclosure can be a peptide, which usually describes a chain of amino acids of from two to about 30 amino acids. The term protein as used herein also describes a chain of amino acids having more than 30 amino acids and can be a fragment or domain of a protein or a full length protein. Furthermore, as used herein, the term protein can refer to a linear chain of amino acids or it can refer to a chain of amino acids that has been processed and folded into a functional protein. It is understood, however, that 30 is an arbitrary number with regard to distinguishing peptides and proteins and the terms can be used interchangeably for a chain of amino acids. The proteins of the present disclosure can be obtained by isolation and purification of the proteins from cells where they are produced naturally, by enzymatic (e.g., proteolytic) cleavage, and/or recombinantly by expression of nucleic acid encoding the proteins or fragments of this disclosure. The proteins and/or fragments of this disclosure can also be obtained by chemical synthesis or other known protocols for producing proteins and fragments.

The term “isolated polypeptide” refers to a polypeptide substantially free of cellular material or culture medium when produced by recombinant DNA techniques, or chemical precursors or other chemicals when chemically synthesized.

The term “vector” as used herein refers to a polynucleotide that can be used to deliver a nucleic acid to the inside of a cell. In one embodiment, a vector is an expression vector comprising expression control sequences (for example, a promoter) operatively linked to a nucleic acid to be expressed in a cell. Vectors known in the art include, but are not limited to, plasmids, phages, cosmids and viruses.

The terms “recipient”, “individual”, “subject”, “host”, and “patient”, are used interchangeably herein and refer to any mammalian subject for whom diagnosis, treatment, or therapy is desired, particularly humans.

As used herein, the terms “treatment,” “treating,” and the like, in some embodiments, refer to administering an agent, or carrying out a procedure, for the purposes of obtaining an effect. The effect may be prophylactic in terms of completely or partially preventing a disease or symptom thereof and/or may be therapeutic in terms of affecting a partial or complete cure for a disease and/or symptoms of the disease. The terms include treatment of a disease or disorder (e.g. inflammation) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. The treatment or amelioration of symptoms is based on one or more objective or subjective parameters; including the results of an examination by a physician. Accordingly, the term “treating” includes the administration of the compounds or agents of the present invention to prevent, delay, alleviate, arrest or inhibit development of the symptoms or conditions associated with diseases (e.g. inflammation).

The term “therapeutic effect” refers to the reduction, elimination, or prevention of the disease, symptoms of the disease, or side effects of the disease in the subject.

The terms “signaling domain” or “intracellular signaling domain” in the context of a CAR-T cell refers to the intracellular domain of the CAR which transduced the activation signal. The signaling domain may be an effector domain that can directly or indirectly promote a biological or physiological response in a cell when receiving the appropriate signal. An effector domain may directly promote a cellular response. An effector domain may also indirectly promote a cellular response by associating with one or more other proteins that promote a cellular response, such as co-stimulatory domains. Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from co-receptor or co-stimulatory molecule. An effector domain can include receptor signaling domains, intracellular signaling components (e.g., cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRa, FcRp (FcsRlb), FcRy, Fyn, HVEM (LIGHTR), ICOS, LAG 3, LAT, Lck, LRP, NKG2D, NOTCHi, pTα, PTCH2, OX40, ROR2, Ryk, SLAMF1, Slp76, TCRα, TCRβ, TRIM, Wnt, Zap70, or any combination thereof. Exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

The terms “is”, “are”, “is derived from” and “are derived from” in the context of a polypeptide or domain of a polypeptide refers to the amino acid sequence of said polypeptide or domain of a polypeptide and indicates that the amino acid sequence is either identical to the native version of said polypeptide or domain of a polypeptide, or a variant of said polypeptide or domain of a polypeptide which is functionally indistinguishable form from the native version of said polypeptide or domain of a polypeptide.

The terms “immune cell” as used herein refers to any cell of hematopoietic lineage involved in regulating an immune response against an antigen (e.g., an autoantigen). In various embodiments, an immune cell is, e.g., a T cell, a B cell, a dendritic cell, a monocyte, a natural killer cell, a macrophage, Langerhan's cells, or Kuffer cells. Preferred immune cells are T cells, such as CD8-positive T cells.

The terms “recombinant” as used herein refers to molecules or cells that are prepared, generated or created by recombinant means, such as genetic engineering or molecular biological technologies. Recombinant molecules do not occur naturally in nature. For example, a recombinant polypeptide or a recombinant nucleic acid refer to a polypeptide (or nucleic acid) which has been modified or which has been put into another context (e.g. by cloning it behind certain regulatory elements, such as a promoter) as compared to the respective wild type molecule. A recombinant immune cell, such as a recombinant T cell, comprises a recombinant polypeptide and/or a recombinant nucleic acid.

The term “cancer” as used herein it its broadest sense refers to diseases in which abnormal cells divide without control.

The term “hematological cancer” as used herein refers to a cancer of the blood, and includes leukemia, lymphoma and myeloma among others. “Leukemia” refers to a cancer of the blood in which too many white blood cells that are ineffective in fighting infection are made, thus crowding out the other parts that make up the blood, such as platelets and red blood cells. It is understood that cases of leukemia are classified as acute or chronic. Certain forms of leukemia include, by way of non limiting examples, acute lymphocytic leukemia (ALL); acute myeloid leukemia (AML); chronic lymphocytic leukemia (CLL); chronic myelogenous leukemia (CML); Myeloproliferative disorder/neoplasm (MPDS); and myelodysplasia syndrome. “Lymphoma” may refer to a Hodgkin's lymphoma, both indolent and aggressive non-Hodgkin's lymphoma, Burkitt's lymphoma, and follicular lymphoma (small cell and large cell), among others. Myeloma may refer to multiple myeloma (MM), giant cell myeloma, heavy-chain myeloma, and light chain or Bence-Jones myeloma.

The term “solid tumor” or “solid cancer” as used herein refers to tumors that usually do not contain cysts or liquid areas. Solid tumors as used herein include sarcomas and carcinomas, such as e.g. breast tumors, ovarian tumors, gastric tumors, lung tumors, pancreatic tumors, prostate tumors, melanoma tumors, colorectal tumors, lung tumors, head and neck tumors, bladder tumors, esophageal tumors, liver tumors, thyroid tumors non-small-cell lung cancer (NSCLC) and kidney tumors.

### FIGURE LEGENDS

FIG. 1 shows the expression levels of miR155 and seven SNPs of miR155 compared to the housekeeping gene U6.

FIG. 2 shows that SNP4 triggers STAT5 signaling significantly stronger compared to wildtype miR155 and to control miR.

FIG. 3 visualizes the down-regulation (panel A) and up-regulation (panel B) of the genes most deregulated in CD8⁺ T cells upon expression of miR155 or SNP4 compared to Ctrl miR.

FIG. 4 shows the effect of the expression of various miR's on the metabolic fitness in T cell. Measured were the extracellular acidification rate (ECAR; left) and the oxygen consumption rate (OCR; right). The arrows indicate when the respective compounds were added.

FIG. 5 shows the cytotoxic potential of CART cells upon expression of various miR's.

FIG. 6 summarizes the functionalities induced by various miR's in T cell. Calculated was the polyfunctional strength index.

FIG. 7 shows in more detail which combination of cytokines are simultaneously produced by subpopulations of cells in Ctrl miR, miR-155 and SNP4 overexpressing CD19-specific CAR T cells. The color intensity shows the percentage of subpopulations in each sample.

FIG. 8 shows CAR T cell mediated anti-tumor immunity in mice, dependent on the expression of various miR's.

### EMBODIMENTS OF THE INVENTION

Nucleic Acids Encoding SNP 4 (rs377265631)

The present invention is based on the surprising finding that one specific SNP of miR155, herein referred to as SNP4, is beneficial in anti-cancer therapy. SNP4 is able to potentiate the anti-tumor activity of other components, such as CAR-T cells. Expression of SNP4 in such cells leads to a potentiation of anti-tumor immunity.

In certain embodiments, the present disclosure related to a recombinant nucleic acid comprising the nucleic acid sequence of SEQ ID No. 2 or SEQ ID No. 5 for use in medicine. The recombinant nucleic acid may be a precursor of the mature miR SNP4, i.e. a nucleic acid comprising the nucleic acid sequence of SEQ ID No. 5. The precursor miR is processed by the cell to yield the mature miR. Hence, in certain embodiments, the present disclosure related to a recombinant nucleic acid comprising the nucleic acid sequence of SEQ ID No. 5 for use in medicine. In other embodiments, the present disclosure related to a recombinant nucleic acid consisting of the nucleic acid sequence of SEQ ID No. 5 for use in medicine. Alternatively. The recombinant nucleic acid may be the mature miR SNP4, i.e. a nucleic acid comprising the nucleic acid sequence of SEQ ID No. 2. Hence, in certain embodiments, the present disclosure related to a recombinant nucleic acid comprising the nucleic acid sequence of SEQ ID No. 2 for use in medicine. In other embodiments, the present disclosure related to a recombinant nucleic acid consisting of the nucleic acid sequence of SEQ ID No. 2 for use in medicine.

In certain embodiments of the present disclosure, the nucleic acid molecules comprising or consisting of the nucleic acid sequence of SEQ ID No. 2 or SEQ ID No. 5 are DNA. In other embodiments of the present disclosure, the nucleic acid molecules comprising or consisting of the nucleic acid sequence of SEQ ID No. 2 or SEQ ID No. 5 are RNA. In preferred embodiments the nucleic acid molecules comprising or consisting of the nucleic acid sequence of SEQ ID No. 2 or SEQ ID No. 5 are DNA. In certain embodiments of the present disclosure, the nucleic acid molecules comprising or consisting of the nucleic acid sequence of SEQ ID No. 2 or SEQ ID No. 5 are modified DNA. In certain embodiments of the present disclosure, the nucleic acid molecules comprising or consisting of the nucleic acid sequence of SEQ ID No. 2 or SEQ ID No. 5 are modified RNA.

In yet other embodiments of the present disclosure, the nucleic acid molecules comprising or consisting of the nucleic acid sequence of SEQ ID No. 2 or SEQ ID No. 5 additional contains regulatory sequences, preferably 5′ to the nucleic acid sequence encoding SNP4.

In certain embodiment of the present disclosure, the nucleic acid molecules comprising or consisting of the nucleic acid sequence of SEQ ID No. 2 or SEQ ID No. 5 are integrated into the genome of a host cell. For integration of the nucleic acid into the genome various technologies are available that are known to the skilled person. Such technologies include Crispr-Cas9 gene editing, as well as base editing or prime editing. For example, since SNP4 differs from miR155 through a single nucleotide exchange from A to G, a wildtype miR155 sequence could be directly converted into SNP4 via an adenine base editor.

In other embodiment of the present disclosure, the nucleic acid molecules comprising or consisting of the nucleic acid sequence of SEQ ID No. 2 or SEQ ID No. 5 are integrated into an extrachromosomal vehicle or vector, such as a plasmid. In yet other embodiment of the present disclosure, the nucleic acid molecules comprising or consisting of the nucleic acid sequence of SEQ ID No. 2 or SEQ ID No. 5 are administered to the cell as free nucleic acid molecules. In such cases the nucleic acid molecules may be administered with additional ingredients which serve to increase the uptake and/or the stability of the nucleic acids.

In certain embodiments of the present disclosure, the nucleic acid molecules comprising or consisting of the nucleic acid sequence of SEQ ID No. 2 or SEQ ID No. 5 is operably linked to a promoter.

In certain embodiments of the present disclosure, the nucleic acid molecules comprising or consisting of the nucleic acid sequence of SEQ ID No. 2 or SEQ ID No. 5 is encoded on an expression vector. In certain embodiments said expression vector is a viral vector or a plasmid. In certain embodiments said expression vector is a viral vector. In certain embodiments said expression vector is a plasmid.

In certain embodiments of the present disclosure, the nucleic acid molecules comprising or consisting of the nucleic acid sequence of SEQ ID No. 2 or SEQ ID No. 5 is an isolated or purified nucleic acid. In certain embodiments of the present disclosure, the nucleic acid molecules comprising or consisting of the nucleic acid sequence of SEQ ID No. 2 or SEQ ID No. 5 is an isolated nucleic acid. In certain embodiments of the present disclosure, the nucleic acid molecules comprising or consisting of the nucleic acid sequence of SEQ ID No. 2 or SEQ ID No. 5 is a purified nucleic acid.

### Immune Cells Expressing SNP4

In certain embodiments, the present disclosure relates to a recombinant immune cell expressing SNP4. The polyfunctional beneficial effect of SNP4 is so directly triggered in the immune cell which is involved in the combat against cancerous tissue. Therefore, in certain embodiments, the present disclosure relates to a recombinant immune cell expressing a nucleic acid comprising the nucleic acid sequence of SEQ ID No. 2 or SEQ ID No. 5. In certain embodiments, the present disclosure relates to a recombinant immune cell expressing a nucleic acid comprising the nucleic acid sequence of SEQ ID No. 2. In other embodiments, the present disclosure relates to a recombinant immune cell expressing a nucleic acid comprising the nucleic acid sequence of SEQ ID No. 5. In yet other embodiments, the present disclosure relates to a recombinant immune cell expressing a nucleic acid consisting of the nucleic acid sequence of SEQ ID No. 2. In yet other embodiments, the present disclosure relates to a recombinant immune cell expressing a nucleic acid consisting of the nucleic acid sequence of SEQ ID No. 5.

The immune cell can be any cell of the hematopoietic lineage involved in regulating an immune response against an antigen. Certain preferred immune cells are T cells. Therefore, in certain embodiments, the present disclosure relates to a recombinant T cell expressing a nucleic acid comprising the nucleic acid sequence of SEQ ID No. 2 or SEQ ID No. 5. In certain embodiments, the present disclosure relates to a recombinant T cell expressing a nucleic acid comprising the nucleic acid sequence of SEQ ID No. 2. In other embodiments, the present disclosure relates to a recombinant T cell expressing a nucleic acid comprising the nucleic acid sequence of SEQ ID No. 5. In yet other embodiments, the present disclosure relates to a recombinant T cell expressing a nucleic acid consisting of the nucleic acid sequence of SEQ ID No. 2. In yet other embodiments, the present disclosure relates to a recombinant T cell expressing a nucleic acid consisting of the nucleic acid sequence of SEQ ID No. 5.

In certain preferred embodiments, the T cell is a T cell which is positive for the T cell marker CD8. Therefore, in certain embodiments, the present disclosure relates to a recombinant CD8-positive T cell expressing a nucleic acid comprising the nucleic acid sequence of SEQ ID No. 2 or SEQ ID No. 5. In certain embodiments, the present disclosure relates to a recombinant CD8-positive T cell expressing a nucleic acid comprising the nucleic acid sequence of SEQ ID No. 2. In other embodiments, the present disclosure relates to a recombinant CD8-positive T cell expressing a nucleic acid comprising the nucleic acid sequence of SEQ ID No. 5. In yet other embodiments, the present disclosure relates to a recombinant CD8-positive T cell expressing a nucleic acid consisting of the nucleic acid sequence of SEQ ID No. 2. In yet other embodiments, the present disclosure relates to a CD8-positive recombinant T cell expressing a nucleic acid consisting of the nucleic acid sequence of SEQ ID No. 5. In certain embodiments said CD8-positive T cell is a tumor infiltrating lymphocyte (TIL) or a peripheral blood lymphocyte (PBL) isolated from a patient afflicted with cancer.

The immune cell expressing SNP4 may also express additional recombinant proteins or polypeptides. For example, the immune cell expressing SNP4 may also express a CAR. Therefore, in certain preferred embodiments the present disclosure relates to a recombinant CAR-T cell expressing a nucleic acid comprising the nucleic acid sequence of SEQ ID No. 2 or SEQ ID No. 5. In certain embodiments, the present disclosure relates to a recombinant CAR-T cell expressing a nucleic acid comprising the nucleic acid sequence of SEQ ID No. 2. In other embodiments, the present disclosure relates to a recombinant CAR-T cell expressing a nucleic acid comprising the nucleic acid sequence of SEQ ID No. 5. In yet other embodiments, the present disclosure relates to a recombinant CAR-T cell expressing a nucleic acid consisting of the nucleic acid sequence of SEQ ID No. 2. In yet other embodiments, the present disclosure relates to a recombinant CAR-T cell expressing a nucleic acid consisting of the nucleic acid sequence of SEQ ID No. 5.

In other embodiments, the immune cell expressing SNP4 may also express a T cell receptor. Therefore, in certain preferred embodiments the present disclosure relates to a recombinant T cell expressing a T cell receptor and a nucleic acid comprising the nucleic acid sequence of SEQ ID No. 2 or SEQ ID No. 5. In certain embodiments, the present disclosure relates to a recombinant T cell expressing a T cell receptor and a nucleic acid sequence of SEQ ID No. 2. In other embodiments, the present disclosure relates to a recombinant T cell expressing a T cell receptor and a nucleic acid comprising the nucleic acid sequence of SEQ ID No. 5. In yet other embodiments, the present disclosure relates to a recombinant T cell expressing a T cell receptor and a nucleic acid sequence of SEQ ID No. 2. In yet other embodiments, the present disclosure relates to a recombinant T cell expressing a T cell receptor and a nucleic acid sequence of SEQ ID No. 5.

In certain embodiments, the immune cell expressing said nucleic acid comprising the nucleic acid sequence of SEQ ID No. 2 or SEQ ID No. 5 is located within the coding region of the chimeric antigen receptor or the T cell receptor, such as the intracellular part of the chimeric antigen receptor or the T cell receptor. In certain embodiments, said chimeric antigen receptor or said T cell receptor is specific for a cancer antigen.

In certain embodiments of the present disclosure, said immune cell is an isolated or purified immune cell. In certain embodiments, said immune cell is an isolated immune cell. In certain embodiments, said immune cell is a purified immune cell. In certain embodiments, said immune cell is a human immune cell.

In certain embodiments, the present disclosure relates to a population of cells comprising at least one immune cell as described herein. In certain embodiments, the present disclosure relates to a composition comprising at least one immune cell as described herein and a pharmaceutical acceptable carrier.

### Therapeutic Use

Expression of SNP4 elicits a beneficial effect in anti-cancer therapy. Expression of SNP4 also leads to the potentiation of the anti-tumor activity of other components, such as CAR-T cells. Therefore, in certain embodiments, the present disclosure relates to the recombinant nucleic acids of the present disclosure for use in the treatment of cancer. In other embodiments, the present disclosure relates to recombinant nucleic acid comprising the nucleic acid sequence of SEQ ID No. 2 or SEQ ID No. 5 for use in the treatment of cancer.

Various types of cancer may be treated with the inventive concept disclosed herein, such as hematological cancer or solid cancer. Therefore, in certain embodiments, the present disclosure relates to the recombinant nucleic acids of the present disclosure for use in the treatment of hematological cancer. In other embodiments, the present disclosure relates to recombinant nucleic acid comprising the nucleic acid sequence of SEQ ID No. 2 or SEQ ID No. 5 for use in the treatment of hematological cancer. In other embodiments, the present disclosure relates to the recombinant nucleic acids of the present disclosure for use in the treatment of solid cancer. In other embodiments, the present disclosure relates to recombinant nucleic acid comprising the nucleic acid sequence of SEQ ID No. 2 or SEQ ID No. 5 for use in the treatment of solid cancer. In other embodiments, the present disclosure relates to recombinant nucleic acid comprising the nucleic acid sequence of SEQ ID No. 2 or SEQ ID No. 5 for use in the treatment of melanoma. In other embodiments, the present disclosure relates to recombinant nucleic acid comprising the nucleic acid sequence of SEQ ID No. 2 or SEQ ID No. 5 for use in the treatment of cervical cancer. In certain embodiments said cervical cancer is HPV-associated cervical cancer. In other embodiments, the present disclosure relates to recombinant nucleic acid comprising the nucleic acid sequence of SEQ ID No. 2 or SEQ ID No. 5 for use in the treatment of a HPV-associated cancer. In other embodiments, the present disclosure relates to recombinant nucleic acid comprising the nucleic acid sequence of SEQ ID No. 2 or SEQ ID No. 5 for use in the treatment of acute lymphocytic leukemia (ALL). In other embodiments, the present disclosure relates to recombinant nucleic acid comprising the nucleic acid sequence of SEQ ID No. 2 or SEQ ID No. 5 for use in the treatment of sarcoma. In other embodiments, the present disclosure relates to recombinant nucleic acid comprising the nucleic acid sequence of SEQ ID No. 2 or SEQ ID No. 5 for use in the treatment of non-small-cell lung cancer (NSCLC).

In certain embodiments, the present disclosure relates to a method of reducing the size of a tumor in a mammal, comprising administering to the mammal a recombinant nucleic acid comprising the nucleic acid sequence of SEQ ID No. 2 or SEQ ID No.5 in an amount effective to reduce the size of the tumor in the mammal.

In certain embodiments, the present disclosure relates to a method of reducing the size of a tumor in a mammal, comprising administering to the mammal a recombinant immune cell comprising a nucleic acid of SEQ ID No. 2 or SEQ ID No. 5. In an amount effective to reduce the size of the tumor in the mammal. In certain embodiments, said immune cell is autologous to the mammal. In other embodiments, said immune cell is allogeneic to the mammal.

In certain embodiments, the present disclosure relates to a method of increasing T cell mediated immunity in a subject having a disease state comprising: isolating a population of the subject's immune cells; introducing a nucleic acid molecule encoding a chimeric antigen receptor or a T cell receptor and nucleic acid comprising the nucleic acid sequence of SEQ ID No. 2 or SEQ ID No. 5 into the isolated immune cells; and reintroducing the immune cells into said subject.

In certain embodiments, the present disclosure relates to a method of increasing CD8+ T cell mediated immunity in a subject having a disease state comprising: isolating a population of the subject's immune cells; introducing a nucleic acid molecule encoding a SNP4 transcript into the isolated immune cells; and reintroducing immune cells into said subject. In certain embodiments, said nucleic acid is introduced into the immune cells by transduction or transfection. In certain embodiments, said immune cell is autologous to the mammal. In certain embodiments, said immune cells are isolated from the blood of the subject.

In certain embodiments, the present disclosure relates to a recombinant immune cell, a population of cells or a composition according to the present disclosure for use in medicine. In certain embodiments, said use in medicine is the use in the treatment of cancer. In certain embodiments, said cancer is a melanoma, NSCLC, sarcoma or a HPV-associated cancers.

### EXAMPLES

### Example 1: Materials and Methods

### Ethics Statement

Peripheral blood mononuclear cells for T-cell enrichment were isolated from peripheral blood of healthy donors. Collection of immune cells from those donors was performed in compliance with the Helsinki Declaration after ethical approval by the local ethical committees (National Institutes of Health Clinical Center, reference number NCT00001846 or Regensburg University, reference number 20-2040-101) and signed informed consent.

### Peripheral Blood Mononuclear Cell Isolation and Pre-Enrichment of Blood Lymphocytes

To isolate T cells from human blood, leukocyte reduction chambers were used. Blood was first diluted with PBS and the resulting blood and PBS mixture was split into four fractions and underlaid with an equal amount of Ficoll-Paque™ Plus (Cytiva, Sweden) Samples were centrifuged at 700×g for 20 minutes at RT, with acceleration set to nine and brake to zero. The PBMC layer was isolated and washed twice with PBS by centrifugation at 300×g for 10 minutes. Naïve CD8 T cells were enriched with EasySep™ Human Naïve CD8+ T Cell Isolation Kit II following manufacturer's protocol.

Generation of CD8⁺ T Cells Overexpressing miRNA

To generate miRNA overexpressing T cells, naïve human CD8+ T cells were enriched with EasySep™ Human Naïve CD8+ T Cell Isolation Kit II (Stemcell Technologies) following manufacturer's protocol. Negatively enriched naïve human CD8⁺ cells were activated with aCD3/CD28 Dynabeads (Thermo Fisher Scientific) for two days in AIM V medium supplemented with 5% FBS (Cytiva), 100 U/ml Penicillin, 100 μg/ml Streptomycin, 2 mM Glutamax, 10 mM HEPES (Thermo Fisher Scientific), and 40 IU/ml IL-2 (Miltenyi biotec). To generate miRNA overexpressing cells activated cells were transduced with a gamma retroviral vector overexpressing either Ctrl miR, miR-155 or SNP4 and NGFR (CD271) selection marker. Following transduction, cells were cultured in 300 IU/mL IL-2 AIM V culture medium for 7 days. Transduced cells were enriched by CD271 EasySep™ Human CD271 Positive Selection Kit II (StemCell Technologies).

Generation of CD19-Specific CAR CD8⁺ T Cells Overexpressing miRNA

To generate miRNA overexpressing CD19-specific CAR T cells, cells were enriched and activated as described above. Two days after activation cells were co-transduced with a gamma retroviral vector overexpressing CD19-CAR construct (J Immunother. 2009 September; 32(7):689-702) and a gamma-retroviral vector overexpressing either Ctrl miR, miR-155 or SNP4 and NGFR selection marker. Transduced cells were cultured in AIMV complete medium supplemented with 300 IU/ml IL-2. Transduced cells were enriched with a CD271 EasySep™ Human CD271 Positive Selection Kit II (StemCell Technologies).

### Mice

Animal studies were carried out under protocols approved by the NCI Bethesda Animal Care and Use Committee. NOD scid γ, NOD.Cg-Prkdcscid Il2rgtm1Wjl/SzJ (NSG) mice were purchased from The Jackson Laboratory. Two million NALM6-GL were injected IV, followed 3 days later by 7.5×10⁵ CD19-specific CAR CD8⁺ T cells overexpressing either Ctrl miR, miR-155 or SNP4. Recombinant human IL-15 (NCI) was injected intraperitoneally every other day (1 μg per mouse). Tumor burden was measured using the Xenogen IVIS Lumina (Caliper Life Sciences). NSG mice were injected intraperitoneally with 3 mg D-luciferin (Caliper Life Sciences) and 4 minutes after injection anesthetized mice were imaged with an exposure time of 30 seconds. Living Image Version 4.1 software (Caliper Life Sciences) was used to analyze the bioluminescent signals for each mouse as photons/s/cm2/sr.

### Cell Lines

NALM6-GL (acute lymphoblastic leukemia cell line, stably transfected with green fluorescent protein and luciferase) and CD19-K562 (chronic myeloid leukemia cells stably expressing CD19 antigen; J Immunother. 2009 September; 32(7):689-702) were cultured in RPMI 1640 medium supplemented with 10% heat-inactivated fetal bovine serum (Cytiva), 100 U/mL penicillin, 100 μg/mL streptomycin, 2 mM glutamax, and 1 mM sodium pyruvate (Thermo Fisher Scientific).

### Flow Cytometry

Cells were filtered with a 40 μm filter unit and acquired on a BD FACSymphony™, a BD FACSCelesta™ or a BD FACSFusion™ flow cytometer (all from Becton Dickinson, Franklin Lakes/NJ, USA). BD CS&T beads were used to validate machine functionality. Fluorescence spillover compensation was performed with AbC™ Total Antibody Compensation Bead Kit (Thermo Fisher Scientific) stained with corresponding antibodies. Flow cytometry data were analyzed using BD FlowJo™ (Version 10.6.2).

Samples were stained either in 5 ml polystyrene tubes or 96-well plates in FACS buffer (1% FCS in PBS). LIVE/DEAD™ Fixable Dead Cell Stain (Thermo Fisher scientific) was used to discriminate dead cells. Following antibodies were used for surface staining of human CD8 T cells samples: anti-CD3 (SK7), anti-CD8 (SK1), anti-NGFR (ME20.4), anti-CD45RA (MEM-56), anti-CD45RO (UCHL1), anti-CD62L (DREG-56), anti-CCR7 (150503), anti-CD25 (BC96), pStat5 (SRBCZX), and anti-CD19 CAR (KIP-1).

### Example 2: MiR155 SNP4 is Overexpressed in Human CD8-Positive T Cells

The expression of miR155 and several SNPs of miR155 after transduction of human CD8 T cells with gamma retroviral vectors expressing either Ctrl miR, miR155 or SNP4, was tested in human CD8⁺ T cells (enriched and transduced as described in Example 1). The SNPs analyzed are shown in the following table:

TABLE 2
(the mature miR is underlined; nucleic acid exchanges in bold and italics):

| | Reference | | SEQ ID |
|---|---|---|---|
| Denominator | name | Sequence of precursor miR | No. |
| miR155 | miR155 | CTGTTAATGCTAATCGTGATAGGGGTTTTTGCCTCCAACTG | 1 |
| | | ACTCCTACATATTAGCATTAACAG | |
| SNP1 | rs76150 | CTGTTAATGCTAATCATGATAGGGGTTTTTGCCTCCAACTG | 6 |
| | 6722 | ACTCCTACATATTAGCATTAACAG | |
| SNP2 | rs14002 | CTGTTAATGCTAATCGTGATAGGGGTTTTTGCCCCCAACTG | 7 |
| | 1681 | ACTCCTACATATTAGCATTAACAG | |
| SNP3 | rs20035 | CTGTTAATGCTAATCGTGATAGGGGTTTTTGCCTCCAACTG | 8 |
| | 1615 | ACTCCTACGTATTAGCATTAACAG | |
| SNP4 | rs37726 | CTGTTAATGCTAATCGTGATAGGGGTTTTTGCCTCCAACTG | 2 |
| | 5631 | ACTCCTACATGTTAGCATTAACAG | |
| SNP5 | rs77465 | CTGTTAATGCTAATCGTGATAGGGGTTTTTGCCTCCAACTA | 9 |
| | 5561 | ACTCCTACATATTAGCATTAACAG | |
| SNP6 | rs75989 | CTGTTAATGCTAATCGTGATAGGGGTTTTTGCCTCCAACTG | 10 |
| | 5287 | ACTCCTATATATTAGCATTAACAG | |
| SNP7 | rs76115 | CTGTTAATGCTAATCGTGATAGGGGTTTTTGCCTCCAACTG | 11 |
| | 1521 | ACTCCTACATATTAGCATTAACAGTGTAC | |

Expression levels were normalized against U6, a commonly used housekeeping non-coding RNA. To quantify miR-155 and U6 we isolated total RNA containing small RNAs from cells with RNeasy Plus Mini Kit (Qiagen). cDNA was synthesized using miRCURY LNA RT kit (Qiagen). qPCR was was performed by miRCURY© LNA© miRNA SYBR© Green PCR Assay (Qiagen). We used hsa-miR-155-5p miRCURY LNA primer set to quantify miR155 and hsa-6-snRNA miRCURY LNA prmer set for U6.

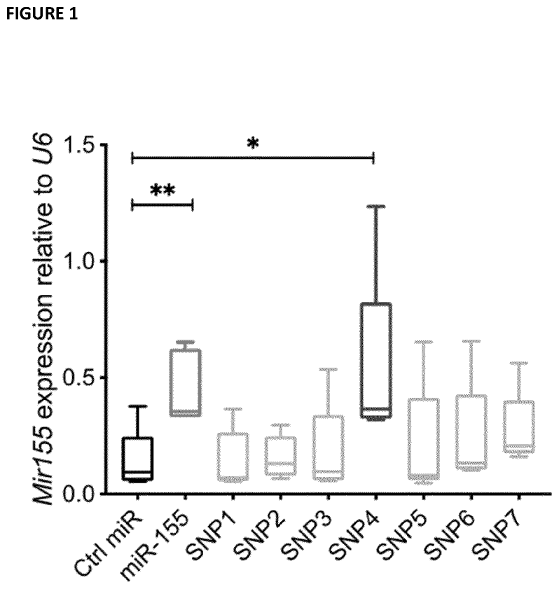

Results are shown in FIG. 1. Among all SNPs tested, SNP4 showed the highest expression level. The average expression level of SNP4 is in the range of wildtype miR155, but SNP4 shows a high degree of variability in expression, in particular with respect to higher expression levels. This finding prompted us to further investigate SNP4 and its role in CD8 T cell biology and anti-tumor function.

### Example 3: SNP4 Triggers Stronger STAT5 Signaling than Wildtype miR155

Next, we investigated the effect of SNP4 on STAT5 signaling compared to wildtype miR155 and to a control miR. As a control miR (Ctrl miR) a scrambled control miR was used (tebubio, France, Cat no. 217CmiR0001-MR04). The Ctrl miR has a scrambled miR sequence:

(SEQ ID NO. 12)
GTAGGTCGACGTTTAAACGCGATCGCAGATCTGCATGTCGATAACGCAGA
GACTCAACACCCTGTTTATTGATGCTGATGAATGACAGCTCGTAATTCAG
TGACTGACTGGCCAGGTTCATCTGCTGTAATAACGCCCCGGACGCGGGAG
TGGCCGAGGCGTTAGCAGAGAATAACAGGCTACCTGTCACTAATGACATG
GCAAACCAAAGTTGCTTCAAAGCTTGATGAATTGAAGCTTTTTTGAATTC

To measure STAT5 phosphorylation human naïve CD8 T cells were transduced as described above. 7 days post-transduction, cells were starved of cytokines for 24 h and then cytokines were added back for either 20 minutes before pStat5 levels were measured by flow cytometry.

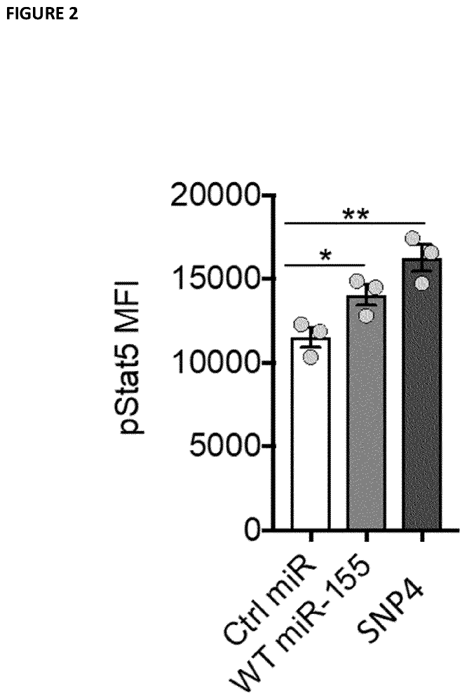

Results are shown in FIG. 2. SNP4 triggered a significantly increased phosphorylation of STAT5 compared to wildtype miR155 and compared to the control miR. This indicates that SNP4 efficiently increases T cell activation in response to cytokine stimulation.

### Example 4: SNP4 De-Regulates miR155 Target Genes More Robust than Wildtype miR155

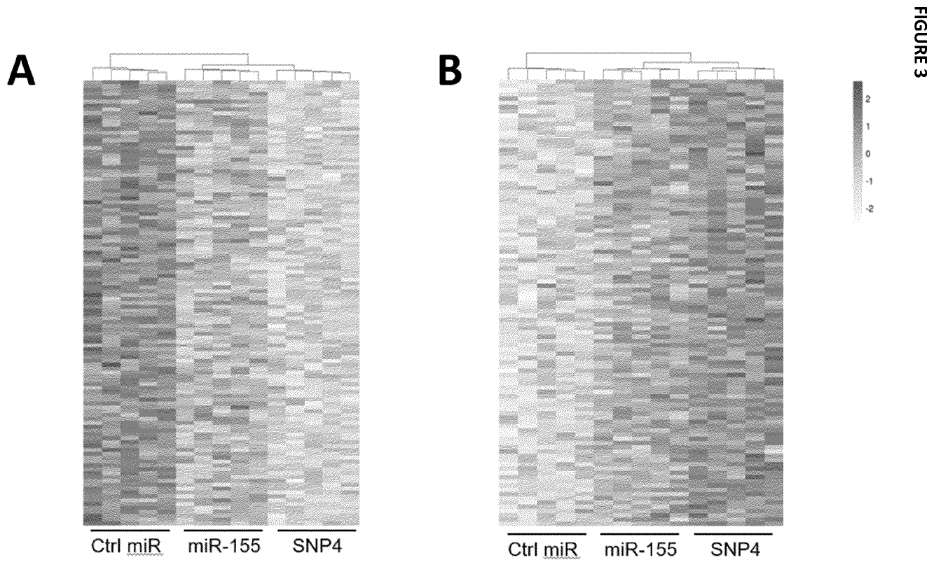

Next, we investigated the transcriptional changes triggered by SNP4 in comparison to miR155 and a control miR (SEQ ID No. 12). Human CD8+ T cells overexpressing either Ctrl miR, miR-155 or SNP4 were generated by transduction of human naïve CD8+ T cells as described above. Transduced cells were enriched on day 6. NGFR enriched cells were lysed with RLT buffer (Qiagen) on day 9 (day 7 post transduction), lysates were frozen at −80° C. and kept until total RNA isolation for total RNA sequencing. The most prominently deregulated genes were selected. The level of deregulation of these genes is visualized in FIG. 3. Interestingly, the genes de-regulated by SNP4 appear to be the same as those that are de-regulated by miR155. Even more interestingly the level of de-regulation (down-regulation, as seen in panel A of FIG. 3 and up-regulation, as seen in panel B of FIG. 3) seem to be more pronounced for SNP4 as compared to miR155. This implies that SNP4 elicits the same transcriptional changes but to a stronger degree, further implying a potential beneficial role of SNP4 expression in cancer treatment.

### Example 5: SNP4 Enhance the Metabolic Fitness of T Cells

In this experiment the metabolic fitness of T cell upon overexpression of wildtype miR155, SNP4 and a Ctrl miR was tested. Naïve CD8⁺ T cells were activated, transduced and cultured as described above. On day 6 post activation transduced cells were enriched with CD271 EasySep™ Human CD271 Positive Selection Kit II (StemCell Technologies). On day 9 post activation metabolism of NGFR enriched cells was studied by Agilent Seahorse XF Glycolisis Stress Test (GST) and Mito-Stress Test (MST). Base medium was prepared by supplementing serum-free DMEM medium with 143 mM NaCl, 3 mg/I Phenol Red, followed by adjusting pH to 7.35. For GST experiments, T cells were resuspended in base medium supplemented with 2 mM L-glutamine. For MST test cells were resuspended in base medium supplemented with 1 mM Sodium Pyruvate and 25 mM glucose.

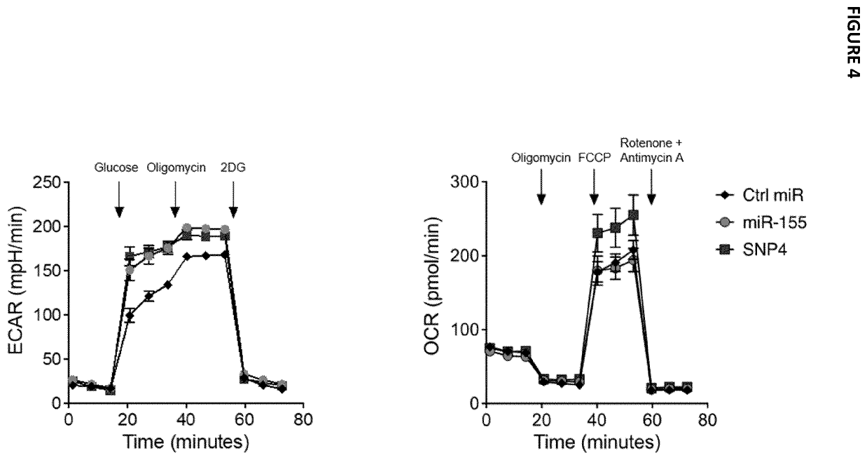

In GST experiments, transduced CD8⁺ T cells were treated with 8 mM glucose, followed by a treatment with 3.9 μM oligomycin and with 122 mM 2DG at time points indicated on the graph (FIG. 4). In MST experiments, cells were treated with 3.75 μM oligomycin, followed by a treatment with 0.35 μM FCCP and with 1.22 μM rotenone plus 1.22 μM antimycin A at time points indicated on the graph (FIG. 4). Measured were the extracellular acidification rate (ECAR) and oxygen consumption rate (OCR), respectively.

Results are shown in FIG. 4. In accordance with their enhanced effector function, cells overexpressing either wt miR-155 or SNP4 were more glycolytic compared to ctrl cells. Interestingly, cells overexpressing SNP4 had higher oxygen consumption rate compared to other two cell types, indicating their higher capacity to produce energy under condition of increased stress. In summary, these data confirm the metabolic advantage of T cells expressing SNP4.

### Example 6: SNP4-Engineered CAR T Cells Exhibit Enhanced Cytotoxic Function

In this experiment it was tested if SNP4 is also able to enhance the cytotoxic potential of CD19-specific CAR T cells. CAR T cells were engineered to express miR155, SNP4 or a Ctrl miR as described above. Six days following activation NGFR transduced cells were enriched by CD271 EasySep™ Human CD271 Positive Selection Kit II (StemCell Technologies). On day 9 post activation miRNA overexpressing CAR T cells were incubated with Nalm6-GL cells in E:T ratio of 1:20 and analyzed by Incucyte Live-Cell Analysis System (Sartorius).

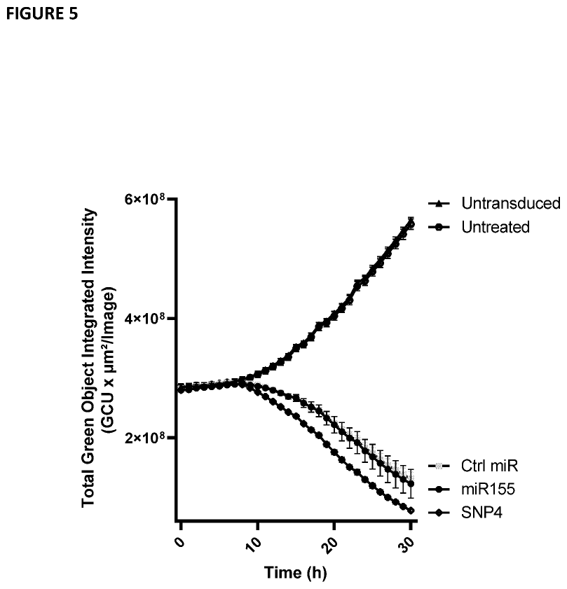

Results are shown in FIG. 5. Nalm6-GL cells co-incubated with untransduced cells continued to grow, as indicated by increasing GFP intensity. GFP intensity decreased in cells co-incubated with T cells transfected with the CAR constructs. The CD19-specific CAR T cells expressing SNP4 showed a significantly stronger cytotoxicity compared to the CD19-specific CAR T cells expressing wildtype miR155 or Ctrl miR.

This experiment demonstrates that SNP4 is able to enhance the cytotoxic function of anti-cancer agents, such as T cells expressing a CAR.

### Example 7: SNP4-Engineered CAR T Cells Exhibit Enhanced Polyfunctionality

In this experiment, polyfunctionality of miRNA overexpressing CAR T cells were tested. Cells were transduced as described above. On day 6 post activation cells were enriched with CD271 EasySep™ Human CD271 Positive Selection Kit II (StemCell Technologies). On day 9 post activation cells were incubated with CD19-K562 target cells for 16 h. After 16 h, target cells were depleted by CD235a magnetic labeling (Miltenyi biotec) and T cells were analyzed by IsoLight technology.

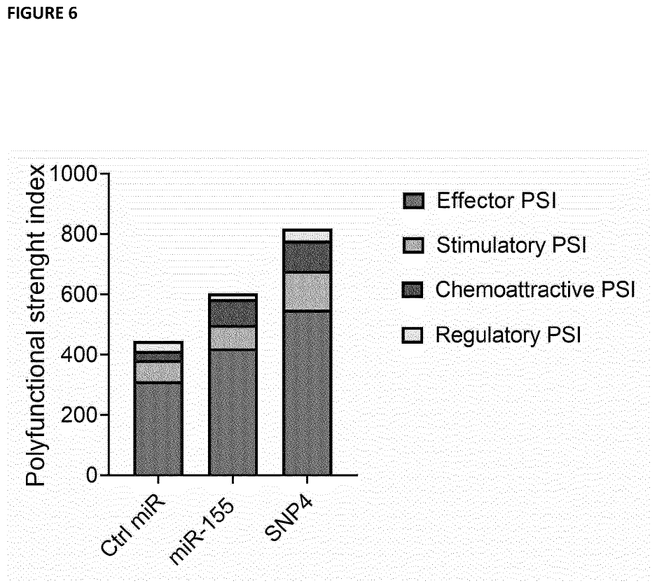

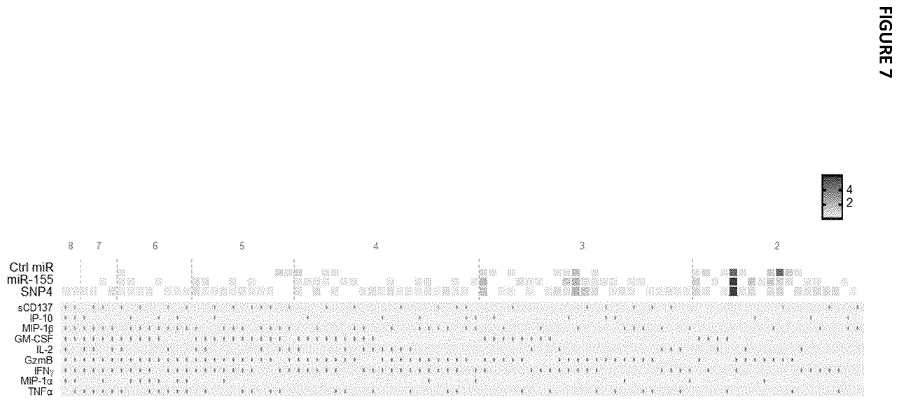

FIG. 6 shows bar charts summarizing the individual functions triggered by the various constructs by way of a polyfunctional strength index that is calculated by multiplying mean fluorescence intensity of secreted cytokines with the percentage of polyfunctional cells. CAR T cells expressing miR155 and SNP4 triggered a strongly increased polyfunctionality compared to CAR T cells expressing Ctrl miR. SNP4 expressing CAR T cells exhibited the highest PSI. FIG. 7 shows combinations of cytokines simultaneously produced by subpopulations of cells present in the three groups of CD19-specific CAR T cells. Overexpression of SNP4 resulted in the highest polyfunctionality of CD19-specific CAR T cells leading to simultaneous production of up to eight different cytokines.

In summary, this experiment confirms the beneficial function of T cells expressing SNP4. It also demonstrates that this is mainly due to stimulatory effects which are triggered by SNP4.

### Example 8: SNP4 Further Enhances CD8⁺ CAR T Cell Antitumor Immunity

In this experiment, it was tested if SNP4 is able to enhance CAR T cell antitumor immunity in mice. To that end human CD8⁺ T cells were co-transduced with gamma retroviral vector overexpressing CD19 CAR and gamma retroviral vector overexpressing either Ctrl miR, miR-155 or SNP4 and NGFR selection marker. NGFR transduced cells were enriched with CD271 EasySep™ Human CD271 Positive Selection Kit II (StemCell Technologies) seven days following transduction. 0.75 million 99% NGFR/70% CD19-specific CD8⁺ CAR cells were injected i.v. three days after 2.0 million NALM6-GL leukemia cells were injected i.v., and 1 μg rhIL-15 cytokine support was administered i.p. every other day for the duration of the experiment.

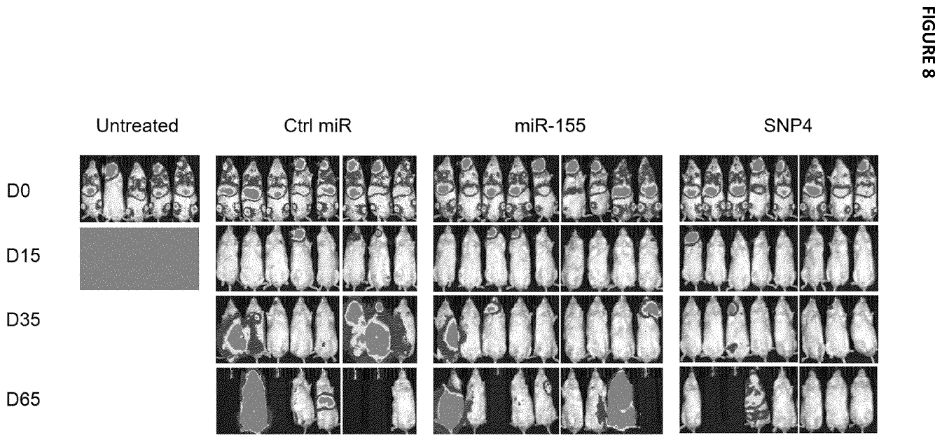

Results are shown in FIG. 8. 15 days post administration all untreated mice were dead while all three CAR T cell types mediated strong anti-tumor response (as shown by decrease of tumor cells-derived bioluminescent signals). After 35 days, mice implanted with CAR T cells expressing miR155 and SNP4 showed a clear decrease in cancerous cells, wherein the decrease is particularly pronounced in the SNP4 mice. After 65 days the effect is even more pronounced and the effect on cancer cells between miR155 mice and SNP4 mice is strongly visible.

1: A recombinant immune cell expressing a nucleic acid of SEQ ID No. 2 or SEQ ID No. 5 for use in medicine.

2: The recombinant immune cell according to claim 1, wherein said nucleic acid is operably linked to a promoter.

3: The recombinant immune cell according to claim 1, wherein said recombinant nucleic acid is encoded on an expression vector, preferably a viral vector or a plasmid.

4: The recombinant immune cell according to claim 3, wherein said expression vectors triggers the high level expression of the nucleic acid of SEQ ID No. 2 or SEQ ID No.5.

5: The recombinant immune cell according to claim 1, where said immune cell further comprises a nucleic acid encoding a chimeric antigen receptor or a T cell receptor.

6: The recombinant immune cell according to claim 5, wherein the nucleic acid comprising the nucleic acid sequence of SEQ ID No. 2 or SEQ ID No. 5 is located within the coding region of said chimeric antigen receptor or said T cell receptor, preferably in the intracellular part of the chimeric antigen receptor or the T cell receptor.

7: The recombinant immune cell according to claim 5, wherein said chimeric antigen receptor or said T cell receptor is specific for a cancer antigen.

8: A method for the treatment of cancer, in a patient, said method comprising administering to the patient the recombinant immune cell according to claim 1.

9: The recombinant immune cell according to claim 1, wherein said recombinant immune cell is a T cell.

10: The recombinant immune cell according to claim 1, wherein said immune cell is an isolated or purified immune cell.

11: A population of cells comprising at least one recombinant immune cell of claim 1.

12: A composition comprising at least one recombinant immune cell of claim 1 and a pharmaceutical carrier.

13: The method of claim 8 wherein the cancer is melanoma, non-small-cell lung cancer, sarcoma or a HPV-associated cancer.

14: The recombinant immune cell according to claim 9, wherein the T cell is a CD8-positive T cell.

15: The recombinant immune cell according to claim 9, wherein the T cell is a tumor infiltrating lymphocyte (TIL) or a peripheral blood lymphocyte (PBL) isolated from a patient afflicted with cancer.

16: The recombinant immune cell according to claim 10, wherein the isolated or purified immune cell is an isolated or purified human immune cell.

17: A composition comprising a population of cells according to claim 11, and a pharmaceutical carrier.
